(19) United States
(12) Patent Application Publication
(10) Pub. No.: US 2014/0134245 A1
(43) Pub. Date: May 15, 2014

(54) IBUPROFEN CHEWABLE TABLET

(71) Applicants: Lei Yang, Tianjin (CN); Fang Yang, Tianjin (CN); John Jiangnan Liu, Shanghai (CN)

(72) Inventors: Lei Yang, Tianjin (CN); Fang Yang, Tianjin (CN); John Jiangnan Liu, Shanghai (CN)

(73) Assignee: Sino-American Tianjin SmithKline and French Lab., LTD, Tianjin (CN)

(21) Appl. No.: 14/126,882

(22) PCT Filed: Jun. 18, 2012
(86) PCT No.: PCT/CN2012/077089

§ 371 Date: Dec. 17, 2013

(30) Foreign Application Priority Data
Jul. 1, 2011 (CN) 201110182886.2

(51) Int. Cl. A61K 9/00; A61K 31/192

(52) U.S. Cl. CPC A61K 9/0056; A61K 31/192
USPC 424/464; 514/570

(57) ABSTRACT

The present invention relates to a taste masked and directly compressed ibuprofen chewable tablet comprising therapeutically effective amount of ibuprofen having average particle size between 250 μm and 400 μm, and a pharmaceutically acceptable carrier.

### FIELD OF THE INVENTION

The present invention relates to a taste masked and directly compressed ibuprofen chewable tablet. Particularly, the present invention relates to a taste masked and directly compressed ibuprofen chewable tablet comprising therapeutically effective amount of ibuprofen having average particle size between 250 μm and 400 μm, and a pharmaceutically acceptable carrier.

### BACKGROUND OF THE INVENTION

Ibuprofen is a widely used analgesic and antipyretic, but is too bitter to be used in chewable tablets for those patients who cannot swallow whole solid-type dosage forms such as tablets or capsules, or demand to relieve the pain such as toothache very quickly. Flavoring agents including but not limited to fruit flavors have been proposed for use with and used with bitter masking drugs. However, flavoring agents are not reliable masking agents for ibuprofen as its bitter properties are very difficult to mask to an appreciable extent.

The traditional methods for masking the taste of ibuprofen in the chewable product have typically involved coating ibuprofen particles with a barrier or coating that will not dissolve in the mouth but will readily dissolve in gastric fluids.

For example, U.S. Pat. No. 5,215,755 described chewable tablets and taste masked granules for making the same, in which the granules were prepared by rotogranulation of the active with polyvinylpyrrolidone, sodium starch glycolate and sodium lauryl sulphate and coated with hydroxylethyl cellulose or a mixture of hydroxyethyl cellulose and hydroxypropylmethyl cellulose. However, this coating remains intact on the ibuprofen granules through normal chewing in the mouth, which delays the onset of ibuprofen active release.

Chinese patent CN100542526C also described an ibuprofen chewable tablet which consists of β-cyclodextrin as inclusion agent and other excipients. However, there is limitation for the effective drug loading due to the ibuprofen-β-cyclodextrin complex formation. Moreover, there is higher cost and lower yield implication associated with the use of β-cyclodextrin.

Chinese patent CN1154481C described a method for preparing a taste masked pharmaceutical composition for compressing into chewable pharmaceutical tablets which comprises: (a) blending together 70 to 90 parts by weight of a pharmaceutically active agent which is water insoluble or only slightly soluble and has an objectionable taste with from 10 to 30 parts by weight of taste masking agent to form a dry blend of said agent and said taste masking agent, wherein said taste masking agent is selected from the group consisting of microcrystalline cellulose, microcrystalline cellulose co-treated with methyl cellulose, and the blend of microcrystalline cellulose and methyl cellulose; (b) adding to the blend, with agitation or stirring, 35 to 65 parts by weight of water for each 100 parts by weight of dry blend to form a wet granulation in which the water is evenly distributed throughout; (c) then forming the wet granulation into taste-masked, spherical particles having a smooth uniform surface and a particle size in the range of 100-1000 μm. The active ingredient in the taste masked pharmaceutical composition may be ibuprofen. The major challenge of this method is the complication related to the handling and equipment requirement of wet-granulation process

Directly compressing into tablet is simpler and more cost efficient, and thus is becoming increasingly important. However, it is known by those skilled in the art that it is challenging to mask the bitterness of ibuprofen by direct compression. Moreover, problems arise during compression of ibuprofen owing to its tendency to adhere because of the relatively low melting point.

In the patent application US2008/0213361, it was described a directly tabletable ibuprofen formulation comprising a) 50-99% by weight of crystalline ibuprofen, b) 1-15% by weight of a finely divided excipient with a surface area of at least 100m²/g, and c) 0-40% by weight of further excipients; wherein the total amount of a), b) and c) corresponds to 100% weight and at least 50% of the surface of the ibuprofen crystals is covered with said finely divided excipient. In this formulation, the ibuprofen crystals have an average particle size of from 20 to 200 μm, and preferably 25-110 μm, implying smaller particle size has superior technical effect. Moreover, it is not disclosed or implied that said ibuprofen formulation is taste acceptable and chewable.

### SUMMARY OF THE INVENTION

The inventors of the present invention surprisingly found that by defining the average particle size between 250 μm and 400 μm, ibuprofen can be easily and directly compressed and the bitterness of ibuprofen can be effectively masked. The present invention was thus made based on this finding.

The present invention provides a taste masked and directly compressed ibuprofen chewable tablet. Particularly, the present invention provides a taste masked and directly compressed ibuprofen chewable tablet comprising therapeutically effective amount of ibuprofen having average particle size of 250 μm and 400 μm, and a pharmaceutically acceptable carrier.

In a preferred embodiment of the present invention, the ibuprofen has the average particle size between 300 μm and 400 μm.

In the taste masked and directly compressed ibuprofen tablet of the present invention, the ibuprofen may preferably be present in an amount of 200-400 mg, more preferably 200-300 mg.

In the taste masked and directly compressed ibuprofen tablet of the present invention, the ibuprofen may particularly be present in an amount of 200 mg.

In the taste masked and directly compressed ibuprofen tablet of the present invention, the ibuprofen may also particularly be present in an amount of 300 mg. In the taste masked and directly compressed ibuprofen tablet of the present invention, the pharmaceutically acceptable carrier may be one or more selected from the group consisting of diluents, binders, disintegrants, stabilizers, sweeteners, flavoring agents, taste enhancing agents, coloring agents, glidants and lubricants.

The taste masked and directly compressed ibuprofen chewable tablet of the present invention may have hardness between 25 N and 100 N, preferably between 40 N and 90 N. More preferably, the ibuprofen chewable tablet of the present invention has the hardness between 55 N and 75 N.

The taste masked and directly compressed ibuprofen chewable tablet of the present invention is taste acceptable, can quickly release ibuprofen active ingredient, and can be manufactured cost effectively.

### DESCRIPTION OF THE INVENTION

The taste masked and directly compressed ibuprofen chewable tablet of the present invention will now be described in terms of its detailed embodiments.

In the taste masked and directly compressed ibuprofen chewable tablet of the present invention, ibuprofen may be used in the form of the free acid or as salt, suitable salts being alkali metal or alkaline earth metal salts or salts with a basic amine or in the form of amino acid salts, for example lysinate salts, in particular sodium ibuprofenate. According to the present invention, the ibuprofen is used in the form of crystalline particles having the average particle size between 250 μm and 400 μm, preferably between 300 μm and 400 μm.

The average particle size of ibuprofen can be determined by various methods known to those skilled in the art. For example, the average size can be determined by sieving analysis and Laser Diffraction Particle Size Analyzer. Preferably, the average particle size is determined by Laser Diffraction Particle Size Analyzer.

Although it is preferred that all particles are within the recited ranges, it is acceptable for small amount of undersized or oversized particles to be present. The ibuprofen having particle size larger than 500 μm can be less than 15% wt, and the ibuprofen having particle size smaller than 150 μm can be less than 10% wt, based on the weight of all ibuprofen particles. The particle size distribution of ibuprofen may be determined, for example, by sieving analysis. It is feasible to ask the ibuprofen manufacturers to produce crystalline particles meeting such requirements.

It is well known to those skilled in the art that the generally used dosages of Ibuprofen are 200 mg, 300 mg and 400 mg. Therefore, in the taste masked and directly compressed ibuprofen chewable tablet of the present invention, the ibuprofen may preferably be present in an amount of 200-400 mg, more preferably 200-300 mg. It is particularly preferred that the ibuprofen is used in an amount of 200 mg or 300 mg in the taste masked and directly compressed chewable tablet of the present invention.

According to the present invention, the pharmaceutically acceptable carriers may be one or more selected from the group consisting of diluents, binders, disintegrants, stabilizers, sweeteners, flavoring agents, taste enhancing agents, coloring agents, glidants and lubricants. It can be appreciated by those skilled in the art that other pharmaceutically acceptable carriers for using in the chewable tablet may also be used to prepare the ibuprofen chewable tablet of the present invention.

Diluents, which include but are not limited to sucrose, mannitol, xylitol, Acesulfame potassium, aspartame, dextrose, fructose, saccharin, sodium saccharin, sorbitol and mixtures thereof can be used. A preferred diluent of the present invention is mannitol, eg. mannitol which is commercially available under the tradename PEARLITOL200SD or PEARLITOL100SD from Roquette (France). The diluents present are preferably in the range of 10 to 90% by weight of tablet.

Diluents of this invention can also serve other functions namely as a sweetener.

The binder in the present invention is used to add cohesiveness to the formulation, thereby providing the necessary bonding to form a cohesive mass or compact upon compression. The binders conventionally used in direct compression tablets includes but not limited to spray dried lactose, compressible starch and those described in Lieberman et al., Pharmaceutical Dosage Forms, 2 Ed., Vol. 1, pp.209-214 (1990), which is hereby incorporated by reference. Preferred binders include but are not limited to cellulose, alkylcelluloses such as methyl cellulose, hydroxyalkyl celluloses such as hydroxypropyl cellulose, low substituted hydroxypropyl cellulose and hydroxypropyl methylcellulose, sodium carboxymethyl cellulose or mixtures thereof, pregelatinised maize starch or polyvinylpyrrolidone. Microcrystalline cellulose, for example commercially available from Asahi Kasei under the tradename AVICEL®PH301 may be used. It is also can be appreciated by those skilled in the art that any new type of binder, eg. Starlac commercially available from Roquette (France), may also be used in the chewable tablet of the present invention.

The binders present are preferably in the range of 0.1 to 30% by weight of tablet.

Disintegrants, which include but are not limited to crospovidone, sodium starch glycolate, starches such as maize starch and dried starch, croscarmellose sodium and cellulose products such as microcrystalline cellulose, microfine cellulose, low substituted hydroxypropylcellulose and the like, either used singly or in admixture can be used. A preferred disintegrant of the present invention is croscarmellose sodium, which is commercially available, for example, from FMC (China).

The disintegrants are preferably present in the range of 0.5 to 20% by weight of tablet.

Stabilizers, which include but are not limited to tribasic sodium phosphate, anhydrous sodium carbonate, glycine, citric acid and the like or mixtures thereof. A preferred stabilizer of the present invention is anhydrous citric acid which is commercially available, for example, from RZBC.

Sweeteners include, but are not limited to natural sweeteners such as sugars e.g. fructose, glucose, sucrose, sugar alcohols such as mannitol, sorbitol or mixtures thereof and artificial sweeteners such as sodium saccharine, sodium cyclamate and aspartame. The sweetening agents are preferably present in the range of 0.1 to 90% by weight of tablet.

Flavoring agents, as used herein, refers to an agent or a mixture of agents that adds flavor to a mixture. Representative flavoring agents include but are not limited to orange flavor, banana flavor, lemon mint flavor, strawberry flavor, grape flavor and cream flavor. A preferred flavoring agent of the present invention is orange flavor, which is commercially available, for example, from Firmenich (China). The flavoring agents present are preferably in the range of 0.1 to 5% by weight of tablet.

Taste enhancing agents include but are not limited to sodium chloride, glycine, citric acid, tartaric acid and the like and mixtures thereof. A preferred taste enhancing agent of the present invention is anhydrous citric acid, which is commercially available, for example, from RZBC. The taste enhancing agents present are preferably in the range of 1 to 10% by weight of tablet.

Coloring agents include but are not limited to titanium dioxide pigments, lake colors and iron oxide pigments.

Glidants include but are not limited to colloidal silicon dioxide, talc powder and surfactants, wherein the surfactant is used alone or as an admixture with one or more glidants. Combinations of colloidal silicon dioxide with one or more surfactants may also be used. A preferred glidant of the present invention is colloidal silicon dioxide which is commercially available, for example, from Deggusa (Germany). The glidants present are preferably in the range of 0.5 to 3% by weight of tablet.

Lubricants include but are not limited to magnesium stearate, calcium stearate, zinc stearate, magnesium oxide, sodium stearyl fumarate, hydrogenated vegetable oil, sodium lauryl stearate, stearic acid, cornstarch, colloidal silicon dioxide, talc, and mixtures thereof. According to the present invention, it is preferred to use Magnesium stearate which is commercially available, for example, from Mallinckrodt Inc (US). Lubricants are present in from about 0.1% to about 6% by weight of tablet.

As the present invention relates to directly compressed ibuprofen chewable tablet, it can be easily appreciated by those skilled in the art to appropriately treat, for example sieve, the above mentioned pharmaceutically acceptable carriers to ensure that the pharmaceutically acceptable carriers can be directly compressed into tablet together with ibuprofen having average particle size between 250 μm and 400 μm.

According to the present invention, the external pressure applied by the tablet press during the compression step is controlled so that the hardness of the tablet is within 25-100 N, preferably 40-90 N, more preferably 55-75 N. It can be appreciated by those skilled in the art that the hardness may be adjusted within the ranges considering the different conditions of the patients. For example, to ensure that the chewable tablet of the present invention can be easily taken by the patients with toothache, the tablet may be compressed softer. The Hardness is measured by conventional hardness testing equipment, such as Tablet Hardness Tester commercially available, for example, from Pharmatron.

### EXAMPLES

The following examples are provided in order to better teach and disclose specific embodiments of the present invention and the manner in which the chewable tablets of the present invention may be prepared. Those skilled in the art will appreciate that the examples are for illustrative purposes only, and that certain variations and changes may be made to alter these formulations and processes. Such variations and changes are still considered to fall within the spirit and scope of the present invention.

### Example 1

### Determination of Average Particle Size and Particle Size Distribution of Ibuprofen

Sieving analysis was used to determine the particle size distribution of ibuprofen. Three batches of ibuprofen particles were manufactured by Hubei Granules-Biocause (China) based on the applicant's requirements, and 25 g of ibuprofen particles of each batch was tested. Sieving device Vibrotronic TypVE1 commercially available from Retsch German and U.S.A Standard Testing Sieves commercially available from FISHER SCIENTIFIC COMPANY were used to sieve the ibuprofen particles with the amplitude of vibration 1.5 mm and the vibration time 20 min. The sieve sizes chosen were 35 mesh (500 μm), 40 mesh (425 μm), 45 mesh (355 μm), 50 mesh (300 μm), 60 mesh (250 μm), 70 mesh (212 μm) and 100 mesh (150 μm).

Laser Diffraction Particle Size Analyzer (Mastersizer 2000) commercially available from Marlven was used to determine the average particle size (D50) of ibuprofen particles.

The average particle size and particle size distribution of Ibuprofen were set forth in Table 1.

TABLE 1
Average particle size and particle size distribution of Ibuprofen.

| | Ibuprofen Batch No. | | |
|---|---|---|---|
| | 1 | 2 | 3 |
| 35 mesh, Residue (%) | 0 | 0.1 | 0.2 |
| 40 mesh, Residue (%) | 3.4 | 2.8 | 3.0 |
| 45 mesh, Residue (%) | 28.9 | 37.8 | 38.5 |
| 50 mesh, Residue (%) | 39.8 | 36.6 | 38.1 |
| 60 mesh, Residue (%) | 20.0 | 14.2 | 11.8 |
| 70 mesh, Residue (%) | 5.7 | 5.0 | 4.9 |
| 100 mesh, Residue (%) | 1.6 | 2.4 | 2.6 |
| Bottom, Residue (%) | 0.5 | 0.9 | 0.8 |
| D50 (μm) | 330 | 340 | 342 |

### Example 2

### Preparation of Ibuprofen Chewable Tablets

Dispensing and sieving

Each ingredient of the batch formulation as set forth in Table 2 was weighed batch by batch. Ibuprofen as described in Example 1, Pearlitol 200SD, Orange flavor, Anhydrous Citric acid and Colloid Silicon dioxide of each batch were sieved through 16 mesh sieve (Russell) one by one. Aspartame, magnesium stearate and FD&C YEIIOW NO. 6 HT 38-42% were sieved through 20, 20 and 50 mesh sieves respectively. The sieved ingredients and the other ingredient not sieved were blended within 24 hrs.

TABLE 2
Formulation of ibuprofen chewable tablets

| | Ibuprefen | Ibuprofen | |
|---|---|---|---|
| | chewable | chewable | |
| Raw materials | (mg/tab) | (kg/batch) | Suppliers |
| Ibuprofen | 200 | 100 | Hubei Granules-biocause |
| PEARLITOL200SD | 225 | 112.5 | Roquette (France) |
| Microcrystalline | 50 | 25 | Asahi Kasei |
| CellulosePH301 | | | |
| Croscarmellose | 25 | 12.5 | FMC (China) |
| sodium | | | |
| Anhydrous Citric acid | 20 | 10 | RZBC |
| Orange flavor | 4 | 2 | Firmenich (China) |
| Aspartame | 4 | 2 | Beijing vitosweet |
| FD&C YEllOW NO. 6 | 0.5 | 0.25 | Shanghai Colorcon |
| HT 38-42% | | | |
| Colloid Silicon dioxide | 9 | 4.5 | Deggusa (Germany) |
| Magnesium Stearate | 2.5 | 1.25 | Mallinckrodt Inc(US) |
| TOTAL | 540.0 | 270 | — |

Blending 50-70% (vol/vol) of 1000 L Bin blender was optimized as powder filling volume for effective blending. The rotating speed of Bin blender was fixed at 8 rpm. The sieved ibuprofen, the sieved PEARLITOL200SD, Microcrystalline CellulosePH301, Croscarmellose sodium, the sieved Anhydrous Citric acid, the sieved Orange flavor, the sieved Aspartame and the sieved FD&C YEIIOW NO. 6 HT 38-42% were blended for 15 min. Then the sieved Magnesium Stearate and Colloid Silicon dioxide were added into the bin and blended for another 6 min. The final blend was discharged into plastic drum, transferred to the compression room, and compressed into tablets within 72 hrs.

Compressing

The blend obtained above was directly compressed into tablets by using Fette P2020 tablet press. The design and control space of critical process parameters, including rotation speed, main compression and pre-compression force were optimized as set forth in Table 3, and the target hardness was set within the range of 55-75N.

TABLE 3
optimal compression parameter range obtained by design of experiment

| | Parameter | | |
|---|---|---|---|
| | Main | | |
| | compression | Press Speed | Pre-compression |
| Product name | force(KN) | (rpm)/(tab/h) | force (KN) |
| Ibuprofen | 10-15 | 40-50/(target: 45) | 1.5-3.5/(target: 2.5) |
| chewable tablet | | | |

### Example 3

### Taste Test

The taste test was done in the presence of a qualified physician on site. A copy of the sensory questionnaire including 11 questions was distributed to 20 subjects aged between 18 and 65. The chewable tablets prepared in Example 2 comprising 200 mg of Ibuprofen were distributed to the subjects. Each subject was required to take one tablet into the mouth and chew for 15 seconds before swallow it. Subjects were then asked to answer the questions in the questionnaire. The average scores obtained for the questions show that the tablets prepared in Example 2 are taste acceptable. Especially for the question (Q11) on how bitter the chewable tablet was, the average score obtained was 1.95 (between “not bitter at all” and “slightly bitter”, see Table 4). This indicates that the bitterness of chewable tablets is acceptable, which is agreed by all 20 subjects. For the question (Q3) on which of the following best describes your overall opinion of the chewable tablet, the average score obtained was 4.65 (between “neither like it nor dislike it” and “like it slightly”, see Table 4); for the question (Q4) on how you would rate the taste of chewable tablet, the average score obtained was 4.7 (between “neither pleasant nor unpleasant and “slightly pleasant”, see Table 4), indicating that the taste of chewable tablets are acceptable.

TABLE 4
Score standards and average score obtained for the key questions

| | |
|---|---|
| Score standard | Average score obtained |

Q3: Which of the following best describes your OVERALL
OPINION of thechewable tablet?

| | | |
|---|---|---|
| Like it extremely | 7 | 4.65 |
| Like it moderately | 6 | |
| Like it slightly | 5 | |
| Neither like it nor dislike it | 4 | |
| Dislike it slightly | 3 | |
| Dislike it moderately | 2 | |
| Dislike it extremely | 1 | |

Q4: How would you rate the TASTE of the Chewable tablet?

| | | |
|---|---|---|
| Very pleasant | 7 | 4.7 |
| Moderately pleasant | 6 | |
| Slightly pleasant | 5 | |
| Neither pleasant nor unpleasant | 4 | |
| Slightly unpleasant | 3 | |
| Moderately unpleasant | 2 | |
| Very unpleasant | 1 | |

Q11: How BITTER was the chewable tablet?

| | | |
|---|---|---|
| Extremely bitter | 5 | 1.95 |
| Very bitter | 4 | |
| Moderately bitter | 3 | |
| Slightly bitter | 2 | |
| Not bitter at all | 1 | |

### Example 4

### Preparation of Chewable Tablets by Using Ibuprofen of Different Average Particle Size Ranges

The purpose of this Example is to evaluate the impact of 3 average particle size ranges, which are 250-300 μm, 300-350 μm and 350-400 μm on the properties of ibuprofen chewable tablets, as well as to confirm that the full range, ie. 250-400 μm, of Ibuprofen particle size is reasonable for the present invention.

Three batches of Ibuprofen within 3 average particle size ranges were manufactured by Hubei Granules-Biocause (China) based on the applicant's requirements, and the particle size distribution and average particle size of these batches of ibuprofen were determined as described in Example 1.

TABLE 5
Average particle size and particle size distribution of three
batches of Ibuprofen

| | Ibuprofen Batch No. | | | |
|---|---|---|---|---|
| | 1 | 2 | 3 | Limit |
| 35 mesh, Residue (%) | 0.16 | 0.16 | 0.18 | ≦15% |
| 100 mesh, Pass (%) | 1.00 | 0.86 | 0.78 | ≦C10% |
| D50 (μm) | 285 | 314 | 369 | — |

The above described batches of ibuprofen particles were then used to prepare three batches of ibuprofen chewable tablets respectively as described in Example 2. Tablets were sampled at the beginning, middle and end of compression process, and then subjected to content uniformity test, dissolution test and tablet assay.

The test results were set forth in Table 6. The content uniformity, dissolution and tablet assay of the samples showed no significant difference from batch to batch, indicating that the full range, ie. 250-400 μm, of Ibuprofen average particle size can be used to prepare the taste masked and directly compressed ibuprofen chewable tablet of the present invention.

TABLE 6
Testing results of chewable tablets prepared by ibuprofen of different average particle size range

| | | Beginning | | | Middle | | | End | | |
|---|---|---|---|---|---|---|---|---|---|---|
| Sampling phase | | 1^{st} batch | 2^{nd} batch | 3^{rd} batch | 1^{st} batch | 2^{nd} batch | 3^{rd} batch | 1^{st} batch | 2^{nd} batch | 3^{rd} batch |
| Tablet | Average (%)/ | 100.2 | 100.8 | 99.8 | 99.3 | 99.9 | 100.2 | 101.7 | 100.2 | 101.4 |
| Content | Range | 96.9-102.2 | 96.8-105.9 | 97.0-102.5 | 96.7-101.1 | 99.1-101.7 | 98.0-103.1 | 98.6-108.4 | 96.7-102.8 | 99.2-106.1 |
| Uniformity | (85.0-115.0%) | | | | | | | | | |
| | A + 1.80 S <− | 3.2281 | 5.7253 | 3.5048 | 3.1900 | 1.4319 | 2.7404 | 7.5103 | 3.5854 | 5.3268 |
| | 15.0 | | | | | | | | | |
| Dissolution | Ibuprofen % | 101 | 100 | 100 | 100 | 101 | 100 | 101 | 100 | 100 |
| | (≧75%) | | | | | | | | | |
| Tablet | Ibuprofen % | 100.9 | 99.5 | 99.8 | 99.4 | 99.8 | 98.6 | 99.6 | 99.8 | 100.0 |
| Assay | (93.0-107.0%) | | | | | | | | | |

| | | | |
|---|---|---|---|
| Single test result | Pass✓ Fail | Pass✓ Fail | Pass✓ Fail |

| | |
|---|---|
| Overall test results | Pass✓ Fail |

1. A taste masked and directly compressed ibuprofen chewable tablet, comprising therapeutically effective amount of ibuprofen having average particle size between 250 μm and 400 μm, and a pharmaceutically acceptable carrier.

2. The taste masked and directly compressed ibuprofen chewable tablet according to claim 1, wherein said ibuprofen have the average particle size between 300 μm and 400 μm.

3. The taste masked and directly compressed ibuprofen chewable tablet according to claim 1, wherein said ibuprofen is in an amount of 200-400 mg.

4. The taste masked and directly compressed ibuprofen chewable tablet according to claim 3, wherein said ibuprofen is in an amount of 200-300 mg.

5. The taste masked and directly compressed ibuprofen chewable tablet according to claim 4, wherein said ibuprofen is in an amount of 200 mg.

6. The taste masked and directly compressed ibuprofen chewable tablet according to claim 4, wherein said ibuprofen is in an amount of 300 mg.

7. The taste masked and directly compressed ibuprofen chewable tablet according to claim 1, wherein said pharmaceutically acceptable carrier is one or more selected from the group consisting of diluents, binders, disintegrants, stabilizers, sweeteners, flavoring agents, taste enhancing agents, coloring agents, glidants and lubricants.

8. The taste masked and directly compressed ibuprofen chewable tablet according to claim 1, which has the hardness between 25 N and 100 N.

9. The taste masked and directly compressed ibuprofen chewable tablet according to claim 8, which has the hardness between 40 N and 90 N.

10. The taste masked and directly compressed ibuprofen chewable tablet according to claim 8, which has the hardness between 55 N and 75 N.
